(19) United States
(12) Patent Application Publication
(10) Pub. No.: US 2017/0326073 A1
(43) Pub. Date: Nov. 16, 2017

(54) JANUS NANOPARTICLE AND METHOD FOR PRODUCING THE SAME

(71) Applicants: CBC CO., LTD., Tokyo (JP); Masahiro MURAKAMI, Osakasayama-shi, Osaka (JP)

(72) Inventor: Masahiro Murakami, Osakasayama-shi (JP)

(73) Assignees: CBC CO., LTD., Tokyo (JP); Masahiro MURAKAMI, Osakasayama-shi, Osaka (JP)

(21) Appl. No.: 15/529,231

(22) PCT Filed: Nov. 25, 2015
(86) PCT No.: PCT/JP2015/083082

§ 371 Date: May 24, 2017

(30) Foreign Application Priority Data
Nov. 25, 2014 (JP) 2014-237362

(51) Int. Cl. A61K 9/51; A61K 47/14; A61K 9/107; A61K 9/28; A61K 47/42; A61K 47/26

(52) U.S. Cl. CPC A61K 9/51; A61K 47/42; A61K 47/26; A61K 9/1075; A61K 9/2886; A61K 47/14

(57) ABSTRACT

It is an object of the present invention to provide a Janus nanoparticle, into which a drug can be encapsulated by a simple method, and a method for producing the same. According to the present invention, provided is a method for producing a Janus nanoparticle having a particle diameter of 0.01 to 5000 μm, which is composed of a lipid and a polymer, wherein the method comprises: a step of emulsifying a solution of one or more types of lipids and one or more types of polymers dissolved in a common solvent in an aqueous solution of a surfactant(s); and a step of removing the common solvent from the obtained emulsion.

### TECHNICAL FIELD

The present invention relates to a heteromorphic nanoparticle (Janus nanoparticle) composed of two or more types of different substances and a method for producing the same.

### BACKGROUND ART

Janus nanoparticle (heteromorphic nanoparticle), one hemisphere of which is composed of a substance A and the other of which is composed of a substance B different from the substance A, can be applied to a variety of fields. Thus, in recent years, Janus nanoparticle promptly has attracted significant attention (Non Patent Literatures 1 to 4). Application examples of the Janus nanoparticle that have been reported so far include an alternative to surfactants (Non Patent Literature 5), an elementary particle of display (Non Patent Literature 6), and magnetic therapy for cancer (Non Patent Literature 7). As methods for preparing Janus nanoparticles, a method of using a multi-fluid nozzle and a method of utilizing phase separation between polymers have been reported (Non Patent Literatures 8 to 10),

### PRIOR ART LITERATURES

### Non Patent Literatures

### Non Patent Literature 1: Perec V et al., Science 315, 1393 (2007)

### Non Patent Literature 2: Chen Q et al., Science 331, 199 (2011)

### Non Patent Literature 3: Reynwaw B J et al., Nature 447, 461 (2007)

### Non Patent Literature 4: Sacanna S et al., Nature 464, 575 (2010)

### Non Patent Literature 5: Ruhland T et al., Langmuir 27, 9807 (2011)

Non Patent Literature 6: Yin S N et at., Adv. Matter. 23, 2915 (2011)
Non Patent Literature 7: Hu S H et al., J. Am. Chem. Soc. 132, 7234 (2010)
Non Patent Literature 8: Takasi Nisisako et al., Adv. Mater. 2006, 18, 1152-1156
Non Patent Literature 9: Zhihong Nie et al., J. Am. Chem. SOC. 2006, 128, 9408-9412
Non Patent Literature 10: Chariya. Kaewsaneha et al., ACS Appl. Mater. Interfaces, 2013, 5 (6), 1857-1869

### SUMMARY OF INVENTION

### Object to be Solved by the Invention

Promotion of absorption of drugs in a biological membrane has been studied for a very long time, and utilization of an absorption promoter, co-administration of an enzyme inhibitor, utilization of a mucosa adhesive polymer and the like have been proposed. Utilization of fine particles is also one of the proposed techniques. It has been considered that a drug is released from a fine particle near the absorbing surface of a biological membrane, so that the enzymatic decomposition of the drug can be suppressed and the amount of the drug absorbed through the membrane can he increased. However, there is no selectivity in terms of the directionality of the drug released from the fine particle. Thus, some drugs are released towards the absorbing surface whereas other drugs are released towards the side opposite to the absorbing surface. In addition, the: thus released drugs are exposed to the risk of decomposition during a period until reaching the absorbing surface. Takada et al. have proposed a hemispherical formulation, in which the release of a drug from the spherical surface is suppressed and the drug release is limited to the release from the plane. While the plane portion of a drug release surface is adhered to the absorbing surface of a biological membrane, the spherical surface suppresses the drug release to the side opposite to the biological membrane, as well as playing as a barrier against enzymes, so that absorption of the drug in the biological membrane can be efficiently carried out. However, it is technically difficult to produce a fine hemispherical formulation.

It is an object of the present invention to provide a Janus nanoparticle, into which a drug can be encapsulated by a simple method, and a method for producing the same.

### Means for Solving the Object

As a result of intensive studies directed towards achieving the aforementioned object, the present inventors have found that a Janus nanoparticle having a particle diameter of 0.01 to 5000 μm, which is composed of a lipid and a polymer, can be produced by emulsifing a solution of one or more types of lipids and one or more types of polymers dissolved in a common solvent, in a liquid containing a surfactant, and then by removing the above-described common solvent from the Obtained emulsion, thereby completing the present invention.

The present invention provides the following invention.

(1) A method for producing a Janus nanoparticle having a particle diameter of 0.01 to 5000 μm, which is composed of a lipid and a polymer, wherein the method comprises: a step of emulsifying a solution of one or more types of lipids and one or more types of polymers dissolved in a common solvent, in a liquid containing a surfactant; and a step of removing the common solvent from the obtained emulsion.
(2) A method for producing a Janus nanoparticle having a particle diameter of 0.01 to 5000 μm, which is composed of a lipid and a polymer, wherein the method comprises: a step of dissolving or suspending one or two or more types of first surfactants in the oil phase and/or water phase of a solution of one or more types of lipids and one or more types of polymers dissolved in a common solvent, and then emulsifying the solution or suspension to prepare a W/O emulsion; a step of emulsifying the obtained W/O emulsion in an aqueous solution of one or two or more types of second surfactants to prepare a W/O/W emulsion; and a step of removing the common solvent from the obtained W/O/W emulsion.
(3) The method according to (1) or (2), which is for use in controlling the distribution of an inner water phase in the Janus nanoparticle.
(4) The method according to (1) or (2), wherein the solution of one or more types of lipids and one or more types of polymers dissolved in a common solvent comprises a drug.
(5) The method according to (2), which uses an aqueous solution of a surfactant as a first surfactant(s), wherein the aqueous solution of the first surfactant(s) comprises a drug.
(6) A method for producing a Janus nanoparticle having a particle diameter of 0.01 to 5000 μm, which is composed of a lipid and a polymer, wherein the method comprises: a step of dispersing fine drug particles or particles formed by compounding one or more types of additives with a drug in a solution of one or more types of lipids and one or more types of polymers dissolved in a common solvent, to prepare an S/O emulsion; emulsifying the obtained S/O emulsion in an aqueous solution. of a surfactant(s) to prepare an S/O/W emulsion; and a step of removing the common solvent from the S/O/W emulsion.
(7) The method according to any one of (4) to (6), wherein the drug is a peptide, a protein, a nucleic acid, or a nucleic acid derivative.
(8) The method according to any one of (1) to (7), wherein an aqueous solution of a surfactant(s) is used, and the volume of the aqueous solution of a surfactant(s) is 0.5 to 10 times greater than that of the solution of one or more types of lipids and one or more types of polymers dissolved in a common solvent.
(9) The method according to any one of (1) to (8), wherein the melting point of the lipid is 30° C. to 45° C.
(10) The method according to any one of (1) to (9), wherein the lipid is a glyceride base comprising saturated C8-C18 triglyceride fatty acid.
(11) The method according to any one of (1) to (10) wherein the polymer is a lactic acid-glycolic acid copolymer, polylactic acid, polyglycolic acid, or Eudragit (registered trademark) RS100.
(12) The method according to any one of (1) to (11), wherein the surfactant is a nonionic surfactant.
(13) The method according to any one of (1) to (12), wherein the surfactant is polyvinyl alcohol.
(14) The method according to any one of (1) to (13), wherein the common solvent is removed from the obtained emulsion over 30 minutes.
(15) A Janus nanoparticle having a particle diameter of 0.01 to 5000 μm, composed of a lipid and a polymer, which is produced by the method according to any one of (1) to (14).

### Advantageous Effects of Invention

According to the Janus nanoparticle and the method for producing the same of the present invention, a formulation for efficiently promoting absorption of a drug in a biological membrane can be provided.

### BRIEF DESCRIPTION OF DRAWINGS

FIG. 1 shows a state in which a Janus nanoparticle comprising a drug layer is allowed to come into contact with a biological membrane.

FIG. 2 shows formation of Janus nanoparticles in Example 1.

FIG. 3 shows formation of Janus nanoparticles in Example 2.

FIG. 4 shows formation of Janus nanoparticles in Example 3.

FIG. 5 shows formation of Janus nanoparticles in Example 4.

FIG. 6 shows formation of Janus nanoparticles in Example 5.

FIG. 7 shows formation of Janus nanoparticles in Example 6.

FIG. 8 shows formation of Janus nanoparticles in Example 7.

FIG. 9 shows formation of Janus nanoparticles in Example 8.

FIG. 10 shows formation of Janus nanoparticles in Example 9.

FIG. 11 shows formation of Janus nanoparticles in Example 10.

FIG. 12 shows formation of Janus nanoparticles in Example 11.

FIG. 13 shows formation of Janus nanoparticles in Example 12.

FIG. 14 shows formation of Janus nanoparticles in Example 13.

FIG. 15 shows formation of Janus nanoparticles in Examples 21 to 24.

FIG. 16 shows formation of Janus nanoparticles in Examples 25 and 26.

FIG. 17 shows formation of Janus nanoparticles in Examples 27 to 30.

FIG. 18 shows formation of Janus nanoparticles in Examples 31 to 34.

FIG. 19 shows formation of Janus nanoparticles in Examples 35 to 38.

FIG. 20 shows formation of Janus nanoparticles in Examples 39 to 42.

FIG. 21 shows formation of Janus nanoparticles in Examples 43 to 45.

FIG. 22 shows formation of Janus nanoparticles in Examples 46 and 47.

FIG. 23 shows formation of Janus nanoparticles in Example 48.

FIG. 24 shows formation of Janus nanoparticles in Example 49.

FIG. 25 shows formation of Janus nanoparticles in Example 50.

FIG. 26 shows formation of Janus nanoparticles in Example 51.

FIG. 27 shows formation of Janus nanoparticles in Example 52.

FIG. 28 shows a 1 mm scale (1 scale: 0.01 mm) when the magnification of an objective lens is ×4×10×60.

### EMBODIMENTS FOR CARRYING OUT THE INVENTION

Hereinafter, the present invention will be described in more detail.

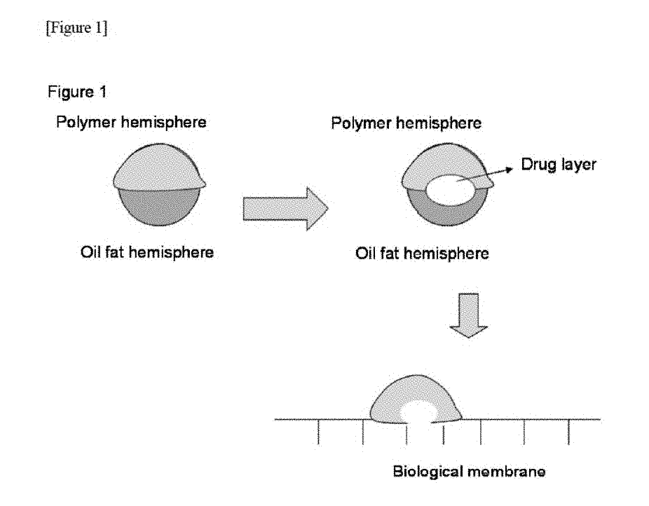

The present inventors have conceived of the system shown in FIG. 1, in which, utilizing the characteristics of a Janus nanoparticle, one hemisphere is configured to suppress the release of a drug to the side opposite to a biological membrane and also to play as a harrier against enzymes, and the other hemisphere is configured to be melted at a temperature close to body temperature and also to be absorbed in a biological membrane when it is allowed to conic into contact with the biological membrane. As a result of creative studies, the present inventors have found that fine particles of interest can be produced by dissolving a polymer and a lipid in a suitable solvent to produce an oil phase, emulsifying the oil phase in a small amount of water phase, and gradually drying it in a liquid, while controlling the time for drying. Moreover, the inventors have also found that, when a surfactant, a water-insoluble solid or a water droplet is added to an oil phase to form a (S/O) emulsion or a water-in-oil (W/O) emulsion, and the thus obtained emulsion is then emulsified in a water phase, a solid or a water droplet can be localized in a fine particle, and a drug layer can be formed. Furthermore, the inventors have found that the distribution of a drug layer in a tine particle can be controlled depending on the type and amount of a surfactant. According to a preferred aspect of the present invention, a spherical fine particle, only a hemisphere of which is melted and absorbed with a temperature rise, can be produced.

The present invention relates to a method for producing a Janus nanoparticle having a particle diameter of 0.01 to 5000 μm, which is composed of a lipid and a polymer, wherein the method comprises: a step of emulsifying a solution of one or more types of lipids and one or more types of polymers dissolved in a common solvent, in a liquid containing a surfactant; and a step of removing the common solvent from the obtained emulsion. Moreover, according to the present invention, there is provided a method for producing a Janus nanoparticle having a particle diameter of 0.01 to 5000 μm, which is composed of a lipid and a polymer, wherein the method comprises: a step of dissolving or suspending one or two or more types of first surfactants in the oil phase and/or water phase of a solution of one or more types of lipids and one or more types of polymers dissolved in a common solvent, and then emulsifying the solution or suspension to prepare a W/O emulsion; a step of emulsifying the obtained W/O emulsion in an aqueous solution of one or two or more types of second surfactants to prepare a W/O/W emulsion; and a step of removing the common solvent from the obtained W/O/W emulsion A Janus nanoparticle having a particle diameter of 0.01 to 5000 μm, composed of a lipid and a polymer, which is produced by the above-described production method of the present invention, is also included in the scope of the present invention.

The particle diameter of a Janus nanoparticle may be 0.01 to 5000 μm, and it is preferably 0.05 to 500 μm, and more preferably 0.1 to 100 μm.

The type of a lipid used in the present invention is not particularly limited. Specific examples of the lipid used herein include saturated fatty acid containing 14 to 24 carbon atoms (e.g., myristic acid, palmitic acid, stearic acid, behenic acid, etc.) or a salt thereof (e.g., a sodium salt and a potassium salt); higher alcohol containing 16 to 24 carbon atoms (e.g., cetyl alcohol, stearyl alcohol, etc.); monoglyceride, diglyceride or triglyceride of saturated and or unsaturated fatty acid containing 8 to 24 carbon atoms; oils and fats (e.g., hydrogenated oils, such as castor oil, cotton seed oil, olive oil, soybean oil, rapeseed oil, or beef tallow); waxes (e.g., beeswax, carnauba wax, spermaceti, etc.); hydrocarbons (e.g., paraffin, microcrystalline wax, etc.); cholesterol; glycolipids (e.g., sphingolipid, ceramide etc.); phospholipids (e.g., phosphatidylcholine, phosphatidylethanolamine, Phosphatidylserine, hydrogenated lecithin, etc.); and hydrophobic sitamins (e.g., vitamin E, vitamin A, vitamin D, vitamin K, etc.). As a lipid, fatty acid triglyceride (tri-O-acylglycerin) that is an ester of fatty acid and glycerin is particularly preferable. These lipids can be used alone or by being appropriately combined and mixed with one another. Furthermore, low melting point lipids, such as C6-C12 lower lipids (e.g., fatty acids such as capric acid or lauric acid or the salts thereof, esters, alcohols, glycolipids, phospholipids, etc.) or vegetable oils (e.g., soybean oil, olive oil, castor oil, rapeseed oil, etc.), are further added to the aforementioned lipids, so that the melting point, consistency or interfacial tension of the oil and fat phase can be appropriately controlled.

The melting point of the lipid is preferably 28° C. to 45° C., more preferably 30° C. to 40° C., further preferably 30° C. to 37° C., and particularly preferably 34° C. to 37° C.

A preferred example of the lipid can be SUPPCIRE AM PASILLES (GATTEFOSSE). SUPPCIRE AM PASILLES is a glyceride base comprising saturated C8-C18 triglyceride fatty acid, which has a melting point of 35.0° C. to 36.5° C. and a hydroxyl value of 5.

The type of a polymer used in the present invention is not particularly limited. Examples of the polymer used herein include polylactic acid, polyglycolic acid, a lactic acid-glycolic acid copolymer, oligolactic acid, polyacrylate, polymethacrylate, an acrylate-methactylate copolymer, polycaprolactone, polyvinyl pyrrolidone (PVP), and a cellulose-based polymer (e.g., ethyl cellulose, hydroxypropylmethyl cellulose (HPMC), hydroxypropyl cellulose (HPC), cellulose acetate phthalate, and hydroxypropylmethyl cellulose phthalate). Examples of the polyacrylate, the polymethacrylate and the acrylate-methacrylate copolymer that can he used herein include an acrylate ammonium methacrylate copolymer (Eudragit (registered trademark) RL100 or RS100, or Eudragit (registered trademark) RL30D or RS30D), an ethyl acrylate methyl methacrylate, copolymer (Eudragit (registered trademark) NE30D), or a methacrylic acid copolymer (Eudragit (registered trademark) L100-55, Eudragit (registered trademark) L30D, Eudragit (registered trademark) E100, and Eudragit (registered trademark) EPO), a starch polymer, and chitosan.

The weight average molecular weight of the polymer is not particularly limited, it is generally approximately 200 to 50,000,000, and preferably approximately 250 to 10,000,000. Among the aforementioned polymers, a lactic acid-glycolic acid copolymer, polylactic acid, polyglycolic acid, and Eudragit (registered trademark) RS100 are particularly preferable.

Eudragit (registered trademark) RS100 is a copolymer of ethyl acrylate, methyl methacrylate and a low content of quaternary ammonium group-containing methacrylic acid ester, and the ammonium group is present in the form of a salt. The structure of Eudragit (registered trademark) RS100 is shown below.

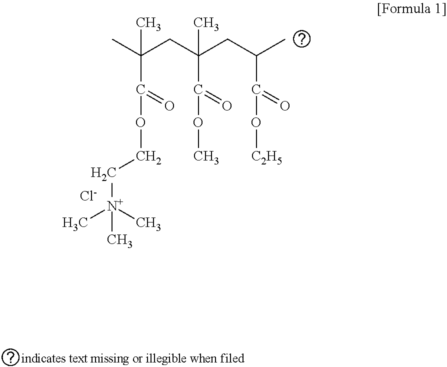 [SMILES: CCOC(=O)C(C)CC(C)(CC(C)(C)C(=O)OCC[N+](C)(C)C)C(=O)OC ; [Cl-]]

In the present invention, a solution of one or more types of lipids and one or more types of polymers dissolved in a common solvent is used. Such a common solvent is not particularly limited, as long as it is a solvent in which both the used lipid and polymer can be dissolved. Examples of the common solvent that can be used herein include methylene chloride, ethyl acetate, hexane, cyclohexane, ethanol, methanol, propanol, acetone, DMF, DMSO, acetic acid, and a mixed solvent of these solvents.

The liquid, which emulsifies the solution of one or more types of lipids and one or more types of polymers dissolved in a common solvent, is not particularly limited, as long as it is a liquid that can be phase-separated from the solution of one or more types of lipids and one or more types of polymers dissolved in a common solvent. Examples of the liquid that can be used herein include water, glycerin, silicon oil, castor oil, soybean oil, and olive oil.

The surfactant used in the present invention (which is also referred to as a “first surfactant” and a “second surfactant”) is not particularly limited, as long as it is capable of emulsifying the above-described solution of a polymer and oil and fat. An anionic surfactant, a cationic surfactant, an amphoteric surfactant, and a nonionic surfactant may be all used herein.

As such a first surfactant, one type of surfactant may be used, or two or more types of different surfactants may also be used in combination. As such two or more types of different surfactants, surfactants each having a different HLB value can be used in combination. In particular, in order to obtain a stable emulsified state, two or more types of surfactants each having a different HLB value can be added to an oil phase and/or a water phase.

Likewise, as a second surfactant, one type of surfactant may be used, or two or more types of different surfactants may also be used in combination. As such two or more types of different surfactants, surfactants each having a different HLB value can be used in combination.

Examples of the anionic surfactant include soap (fatty acid sodium) RCOO⁻Na⁺, monoalkyl sulfate ROSO₃⁻M⁺, alkyl polyoxyethylene sulfate RO(CH₂CH₂O)ₘSO₃⁻M⁺, alkylbenzene sulfonate RR′H₂CHC₆H₄SO₃⁻M⁺, and monoalkyl phosphate ROPO(OH)O⁻M⁺.

Examples of the cationic surfactant include alkyl trimethyl ammonium salt RN⁺(CH₃)₃X⁻, dialkyl dimethyl ammonium salt RR′N⁺(CH₃)₂X⁻, and alkyl benzyl dimethyl ammonium salt RN⁺(CH₂Ph)(CH₃)₂X⁻.

Examples of the amphoteric surfactant include alkyl dimethylamine oxide R(CH₃)₂NO and alkyl carboxybetaine R(CH₃)₂N⁺CH₂COO⁻.

Examples of the nonionic surfactant include polyoxyethylene alkyl ether RO(CH₂CH₂O)ₘH, fatty acid sorbitan ester, alkyl polyglucoside, fatty acid diethanolamide RCON(CH₂CH₂OH)², alkyl monoglyceryl ether ROCH₂CH(OH)CH₂OH, and polyvinyl alcohol.

The surfactant is particularly preferably a nonionic surfactant, and is further particularly preferably polyvinyl alcohol.

The volume of an aqueous solution of a surfactant(s) is preferably 0.5 to 10 times, and more preferably 1 to 10 times greater than the volume of the solution of a lipid and a polymer dissolved in a common solvent.

In the present invention, pharmaceutical additives and a drug can he comprised in the solution of one or more types of lipids and one or more types of polymers dissolved in a common solvent, or in the aqueous solution of the first surfactant(s).

Examples of the drug include a peptide, a protein, a nucleic acid (DNA, a DNA decoy, a DNA plasmid, an aptamer, RNA, SsiRNA, tRNA, miRNA a heteroduplex nucleic acid of DNA and RNA, etc.) or a nucleic acid derivative (including a chimeric compound comprising DNA, RNA, and a derivative thereof), and a peptide nucleic acid.

Specific examples of such a peptide, a protein, a nucleic acid or a nucleic acid derivative, which can be used as a drug herein, include proteins, such as a growth hormone releasing factor, a growth factor, an epidermal growth factor (EGF), a nerve growth factor (NO), TGF, PDGF, an insulin growth factor (IGF), a fibroblast growth factor (aFGF, bFGF, etc.), somatostatin, calcitonin, insulin, vasopressin, interferon, IL-2, urokinase, senatiopeptidase, superoxide dismutase (SOD), thyrotropin releasing hormone (TRH), luteinizing hormone releasing factor (LH-RH), corticotropin releasing hormone (CRF), growth hormone releasing hormone (GHRH), oxytocin, erythropoietin (EPO), or a colony-stimulating factor (CSF), peptides obtained from the portions of such proteins, and peptide vaccines obtained from specific antigens. In addition, nucleic acids encoding these proteins or peptides and the nucleic acid derivatives thereof can also be used.

The nucleic acid derivative means a molecule obtained by modifying a ribonucleotide, a deoxyribonucleotide, RNA or DNA, and such a molecule may be either a naturally existing molecule or a non-natural molecule.

An example of the nucleic acid derivative can be a molecule formed by adding another chemical substance to a nucleic acid. Specific examples of such a nucleic acid derivative include 5′-polyamine-added derivative, a cholesterol-added derivative, a steroid-added derivative, a bile acid-added derivative, a vitamin-added derivative, a Cy5-added derivative, a Cy3-added derivative, a 6-FAM-added derivative, and a biotin-added derivative.

Other examples of the nucleic acid derivative as a sugar modification include oligonucleotide derivatives substituted with 2′-O-propylribose, 2′-methoxyethoxyribose, 2′-O-methylribose, 2′-O-methoxyethylribose, 2′-O-[2-(guanidium)ethyl]ribose, or 2′-O-fluororibose. Moreover, examples of the nucleic acid derivative as a phosphoric acid group modification include an oligonucleotide derivative in which a phosphoric acid diester bond in the oligonucleotide is converted to a phosphorothioate bond, and an oligonucleotide derivative in which a phosphoric acid diester bond in the oligonucleotide is converted to an N3′-P5′ phosphoramidate bond.

In the present invention, the time required for the removal of a common solvent is not particularly limited, as long as desired Janus nanoparticles can be produced therein. It is preferable to remove such a common solvent over 30 minutes or more. The time required for the removal of such a common solvent is more preferably 30 minutes to 6 hours, and further preferably 1 to 4 hours.

A Janus nanoparticle having a particle diameter of 0.01 to 5000 μm, composed off lipid. and a polymer, which is produced by the above-described method of the present invention, is also included in the scope of the present invention.

In the present invention, the solution of one or more types of lipids and one or more types of polymers dissolved in a common solvent, or the aqueous solution of the first surfactant(s) can comprise, as a drug and pharmaceutical additives, a mucolytic agent (e.g., L-cysteine, N-acetylcysteine, etc.), a permeation promoter or an absorption promoter (e.g., medium-chain fatty acid, long-chain unsaturated fatty acid, their monoglycerides, their polyethylene glycol esters, or mixtures of them such as LABRASOL, bile salts, chelating agents, glycolipids including alkyl saccharide as a typical example, Azone (registered trademark), TRANSCUTOL (registered trademark), cyclodextrin, tight junction modifiers such as a Claudin binder and a derivative thereof, etc.), a buffer (e.g., a phosphate buffer, a citrate buffer, a carbonate buffer, etc.), a degrading enzyme inhibitor (e.g., aprotinin, bacitracin, camostat, citric acid, tartaric acid, sodium edetate, sodium glycocholate, SUPERase-In (registered trademark), RNasin (registered trademark) , etc.), a specific gravity adjuster (e.g., glycerin, sucrose, glucose, amino acid, porous silica, etc.), an antioxidant (e.g., ascorbic acid, hydroxybutyl anisole, tocopherol, ascorbic acid, CoQ10, fallerene, a fullerene derivative, etc.), and light-shielding and/or light-absorbing agents (titanium oxide, oxybenzone, octocrylene, etc.).

As an example, the above-described Janus nanoparticle of the present invention can fie used as a Janus type drug-carrying fine particle formulation comprising a hydrophobic permeation promoter and a water-soluble drug.

The present invention will be more specifically described in the following examples. However, these examples are not intended to limit the scope of the present invention.

### EXAMPLES

In the following examples, 0.5% polyvinyl alcohol, 0.1% albumin, and 1% HCO60 were each used in the form of an aqueous solution.

### Example 1

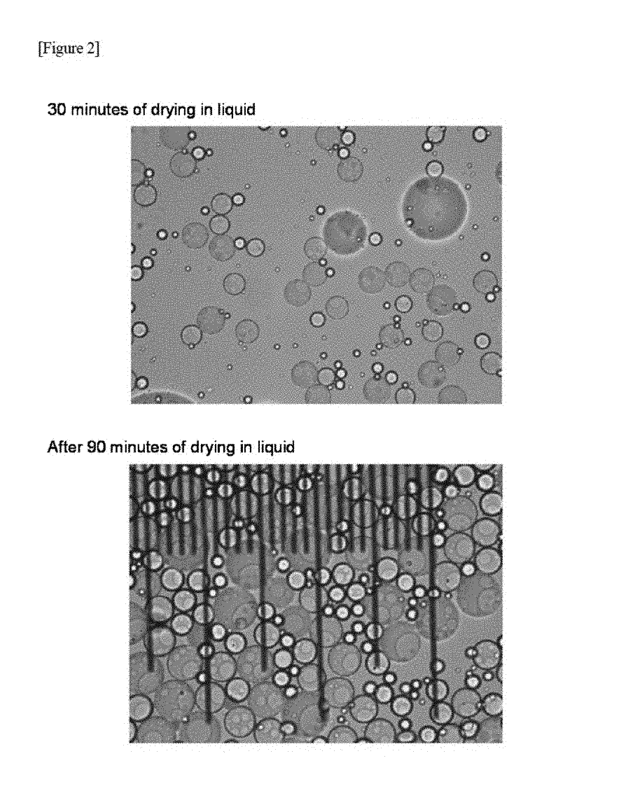

50 mg of a lactic acid-glycolic acid copolymer (Wako Pure Chemical Industries, Ltd.; LGA5020), 100 mg of polylactic acid (Wako Pure Chemical Industries, Ltd.; LA0020), and 100 mg of SUPPCIRE AM PASILLES (GATTEFOSSE) were dissolved in 1 mL of methylene chloride, and the thus prepared solution was then emulsified in 5 mL of 0.5% polyvinyl alcohol (Kuraray Ltd.; KURARAY POVAL 220C) at 5000 rpm for 3 minutes, using a homogenizer (IKA ULTRA-TURRAX 8). After completion of the emulsification, the obtained emulsion was stirred using a stirrer, so that the solvent was distilled away. The remaining emulsion was sampled over time, and was observed under an optical microscope. Morphological changes over time are shown in FIG. 2. Phase separation of droplets was found in the emulsion, and the droplets were then gathered, so that Janus nanoparticles could be formed 3 hours later. Four hours later, some Janus nanoparticles, in which two hemispheres were dissociated from each other, were observed. According to the observation under the optical microscope, the particle diameter of the Janus nanoparticles was found to be 1 to 100 μm.

### Example 2

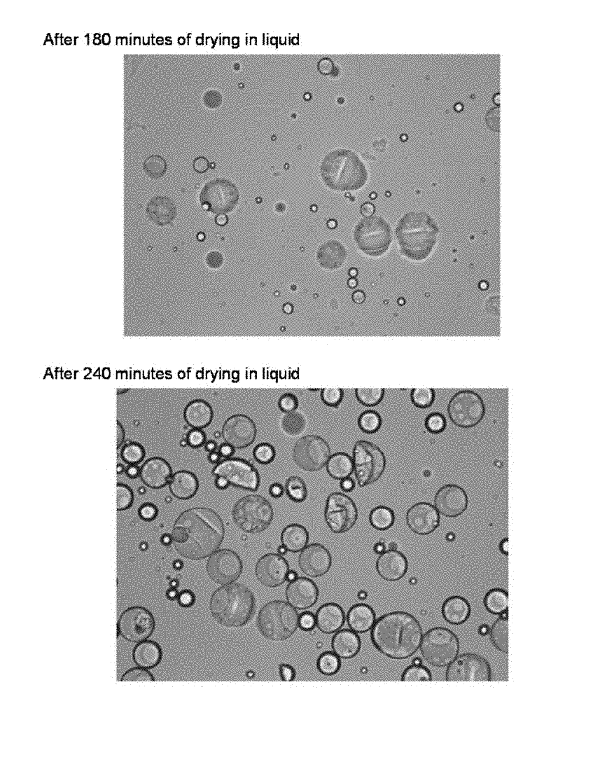

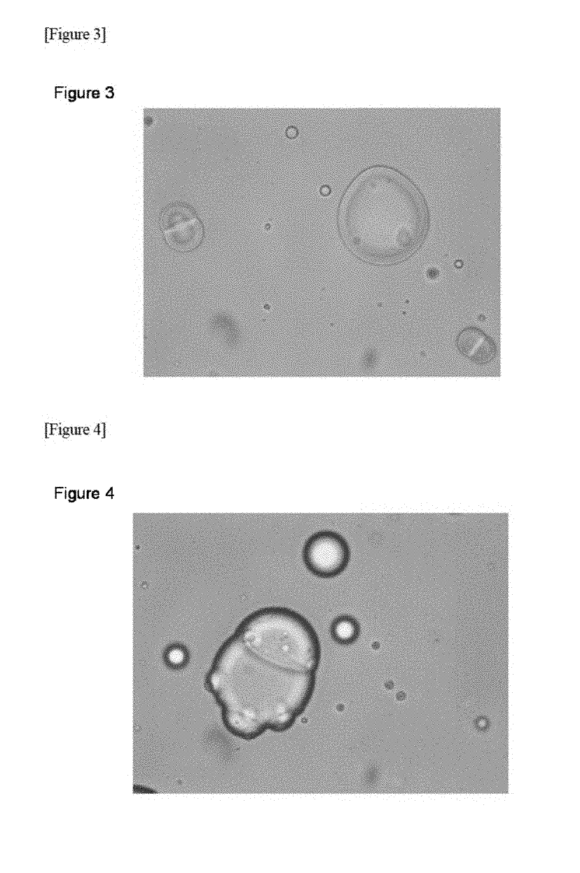

100 mg of a lactic acid-glycolic acid copolymer (Wako Pure Chemical Industries, Ltd.; LGA5020) and 100 mg of SUPPCIRE AM PASILLES (GATTEFOSSE) were dissolved in 1 mL of methylene chloride, and the thus prepared solution was then emulsified in 5 mL of 0.5% polyvinyl alcohol (Kuraray Co., Ltd.; KURARAY POVAL 220C) at 5000 rpm for 3 minutes, using a homogenizer (IKA ULTRA-TURRAX T18). After completion of the emulsification, the obtained. emulsion was stirred using a stirrer, so that the solvent was distilled away. Three hours later, the remaining emulsion was sampled, and was observed under an optical microscope. Fine particles, in which a borderline was observed between hemispheres, were found (FIG. 3). According to the observation under the optical microscope, the particle diameter of the Janus nanoparticles was found to be 1 to 100 μm.

### Example 3

100 mg of a lactic acid-glycolic acid copolymer (Wako Pure Chemical Industries, Ltd.; LGA5020) and 150 mg of SUPPCIRE AM PASILLES (GATTEFOSSE) were dissolved in 1 mL of methylene chloride, and the thus prepared solution was then emulsified in 5 mL of 0.5% polyvinyl alcohol (Kuraray Co., Ltd.; KURARAY POVAL 2.20C) at 5000 rpm for 3 minutes, using a homogenizer (IKA ULTRA-TURRAX T18). After completion of the emulsification, the obtained emulsion was stirred using a stirrer, so that the solvent was distilled away. Three hours later, the remaining emulsion was sampled, and was observed under an optical microscope. Fine particles, each of which was divided into three or more parts, were found (FIG. 4). According to the observation under the optical microscope, the particle diameter of the Janus nanoparticles was found to be 1 to 100 μm.

### Example 4

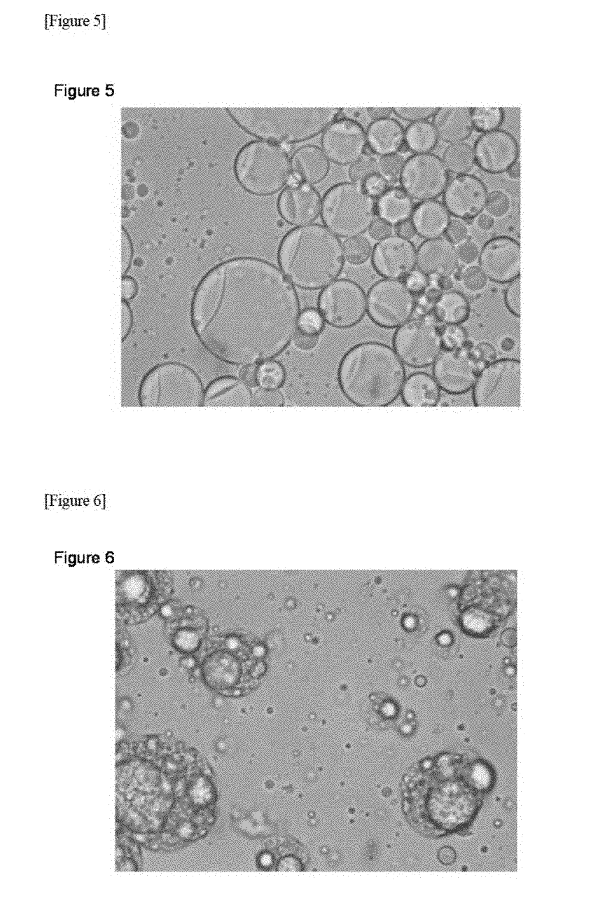

100 mg of a lactic acid-glycolic acid copolymer (Wako Pure Chemical Industries, Ltd.; LGA5020) and 100 mg of SUPPCIRE AM PASILLES (GATTEFOSSE) were dissolved in 3 mL of ethyl acetate, and the thus prepared solution was then emulsified in 5 mL of 0.5% polyvinyl alcohol (Kuraray Co., Ltd.; KURARAY POVAL 220C) at 5000 rpm for 3 minutes, using a homogenizer (IKA. ULTRA-TURRAX T18). After completion of the emulsification, the obtained emulsion was stirred using a stirrer, so that the solvent was distilled away. Three hours later, the remaining emulsion was sampled, and was observed under an optical microscope. Almost all fine particles were found to consist of two parts (FIG. 5). Thereafter, the emulsion was stirred in 200 ml of purified water for 30 minutes, so that the solvent remaining in the fine particles were rapidly extracted. The obtained dispersion was subjected to filtration under reduced pressure using a 20-μm wire mesh filter, so that the fine particles were collected and were then freeze-dried. According to the observation under the optical microscope, the particle diameter of the Janus nanoparticles was found to be 1 to 100 μm.

### Example 5

100 mg of Eudragit RS100 and 100 mg of SUPPCIRE AM PASILLES (GATTEFOSSE) were dissolved in 1 mL of methylene chloride, and the thus prepared solution was then emulsified in 5 mL of 0.5% polyvinyl alcohol (Kuraray Co., Ltd.; KURARAY POVAL 220C) at 5000 rpm for 3 minutes, using a homogenizer (IKA ULTRA-TURRAX T18). After completion of the emulsification, the obtained emulsion was stirred using a stirrer, so that the solvent was distilled away. Three hours later, the remaining emulsion was sampled, and was observed under an optical microscope. Inside fine particles, droplets were unevenly observed near the surfaces of the fine particles (FIG. 6). According to the observation under the optical microscope, the particle diameter of the Janus nanoparticles was found to be 1 to 100 μm.

### Example 6

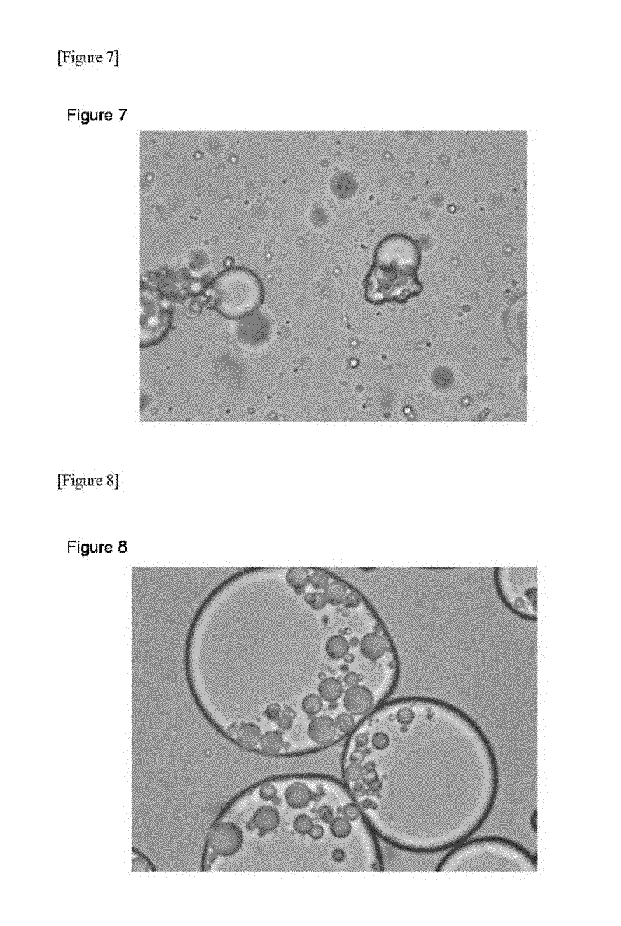

100 mg of a lactic acid-glycolic acid copolymer (Wake Pure Chemical Industries, Ltd.; LGA5020) and 100 mg of SUPPCIRE AM PASILLES (GATTEFOSSE) were dissolved in 1 mL of methylene chloride, and chitosan (Wake Pure Chemical Industries, Ltd.; Chitosan 100) was then dispersed in the thus prepared solution. The obtained dispersion was then emulsified in 5 mL of 0.5% polyvinyl alcohol (Kuraray Co., Ltd.; KURARAY POVAL 220C) at 5000 rpm for 3 minutes, using a homogenizer (IKA ULTRA-TURRAX T18). After completion of the emulsification, the obtained emulsion was stirred using a stirrer, so that the solvent was distilled away. Three hours later, the remaining emulsion was sampled, and was observed under an optical microscope. Fine particles, in some of which swollen chitosan was distributed, were obtained (FIG. 7). According to the observation under the optical microscope, the particle diameter of the Janus nanoparticles was found to be 1 to 100 μm.

### Example 7

100 mg of a lactic acid-glycolic acid copolymer (Wako Pure Chemical Industries, Ltd.; LGA5020) and 100 mg of SUPPCIRE PASILLES (GATTEFOSSE) were dissolved in 1 mL of methylene chloride, and 0.1% albumin (Sigma-Aldrich from chicken egg) was then emulsified in the thus prepared solution at 20,000 rpm for 1 minute, using a homogenizer (IKA ULTRA-TURRAX T18), to prepare a W/O emulsion. The prepared W/O emulsion was emulsified in 5 mL of 0.5% polyvinyl alcohol (Kuraray Co., Ltd.; KURARAY POVAL 220C) at 5000 rpm for 3 minutes, using a homogenizer (IKA ULTRA-TURRAX T18), to prepare a W/O/W emulsion. After completion of the preparation, the W/O/W emulsion was stirred using a stiffer, so that the solvent was distilled away. Three hours later, the remaining emulsion was sampled, and was observed under an optical microscope. In some fine particles, a borderline was observed between the hemispheres thereof, and also, a state in which droplets were distributed in one hemisphere could be observed (FIG. 8). According to the observation under the optical microscope, the particle diameter of the Janus nanoparticles was found to be 1 to 100 μm. The emulsion was left at rest for 30 minutes, and thereafter, the supernatant was removed, and the remaining emulsion was then washed with water and freeze-dried.

### Example 8

100 mg of a lactic acid-glycolic acid copolymer (Wako Pure Chemical Industries, Ltd.; LGA5020), 100 mg of SUPPCIRE AM PASILLES (GATTEFOSSE) and 20 mg of glycerin stearate were dissolved in 1 mL of methylene chloride, and 1% HCO60 was then emulsified in the thus prepared solution at 20,000 rpm for 30 seconds, using a homogenizer (IKA ULTRA-TURRAX T18), to prepare a W/O emulsion. The prepared W/O emulsion was emulsified in 5 mL of 0.5% polyvinyl alcohol (Kuraray Co., Ltd.; KURARAY POVAL 220C) at 5000 rpm for 3 minutes, using a homogenizer (IKA ULTRA-TURRAX T18), to prepare a W/O/W emulsion.

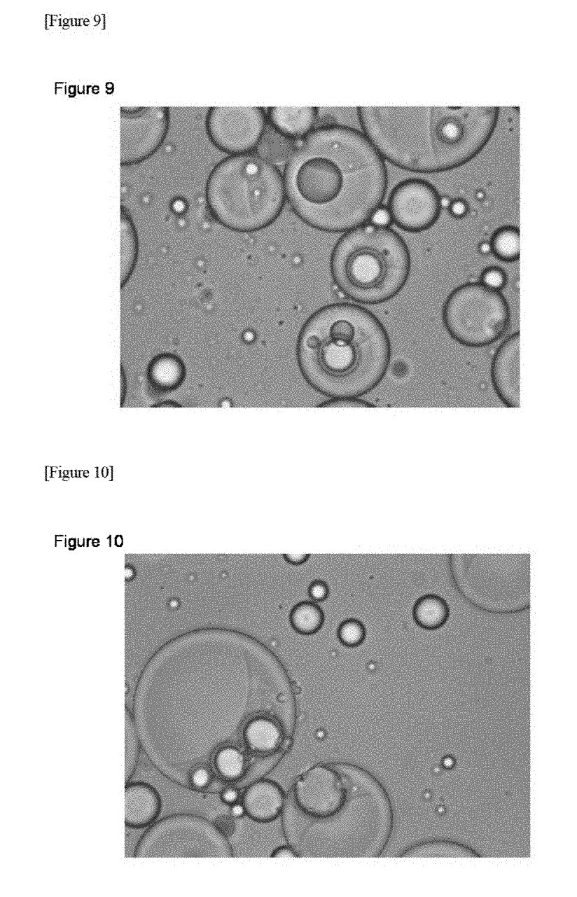

After completion of the preparation, the W/O/W emulsion was stirred using a stirrer, so that the solvent was distilled away. Two hours later, the remaining emulsion was sampled, and was observed under an optical microscope. In some fine particles, a borderline was observed between the hemispheres thereof, and also, a state in which droplets were distributed on the borderline could be observed (FIG. 9). According to the observation under the optical microscope, the particle diameter of the Janus nanoparticles was found to be 1 to 100 μm.

### Example 9

100 mg of a lactic acid-glycolic acid copolymer (Wako Pure Chemical Industries, Ltd.; LGA5020) and 100 mg of SUPPCIRE AM PASILLES (GATTEFOSSE) were dissolved in 1 mL of methylene chloride, and 1% HCO60 was then emulsified in the thus prepared solution at 20,000 rpm for 30 seconds, using a homogenizer (IKA ULTRA-TURRAX T18), to prepare a W/O emulsion. The prepared W/O emulsion was emulsified in 5 mL of 0.5% polyvinyl alcohol (Kuraray Co., Ltd.; KURARAY POVAL 220C) at 5000 rpm for 3 minutes, using a homogenizer (IKA ULTRA-TURRAX T18), to prepare a W/O/W emulsion. After completion of the preparation, the W/O/W emulsion was stirred using a stirrer, so that the solvent was distilled away. Two hours later, the remaining emulsion was sampled, and was observed under an optical microscope. In some fine particles, a borderline was observed between the hemispheres thereof, and also, a state in which droplets were distributed in one hemisphere could be observed (FIG. 10). According to the observation under the optical microscope, the particle diameter of the Janus nanoparticles was found to be 1 to 100 μm.

### Example 10

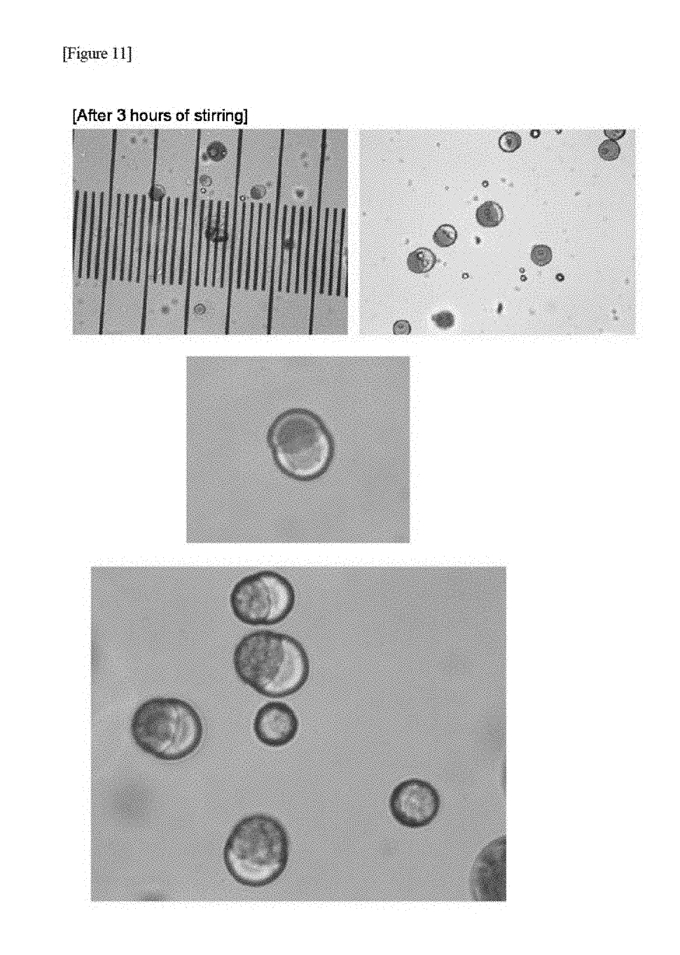

A Janus nanoparticle containing oil red used as a hydrophobic drug model was prepared according to the production method described in Example 1, and discrimination of a hydrophobic region was then confirmed. As a result, as shown in FIG. 11, oil red was localized in a region that was assumed to be a lipid layer, and thus, it was demonstrated that the prepared tine particle was a Janus-type fine particle consisting of two regions having different properties. In addition, the prepared Janus-type fine particle formulation tended to show that the particles are gathered such that hydrophobic region surfaces, in which the oil red is present, are inwardly contacted with each other. On the other hand, even after the present Janus-type fine particle formulation has bee left at rest overnight, the shape of the Janus-type fine particles is maintained, and thus, it became clear that re-dispersion would be possible. According to the observation under an optical microscope, the particle diameter of the Janus nanoparticles was found to be 1 to 100 μm.

### Example 11

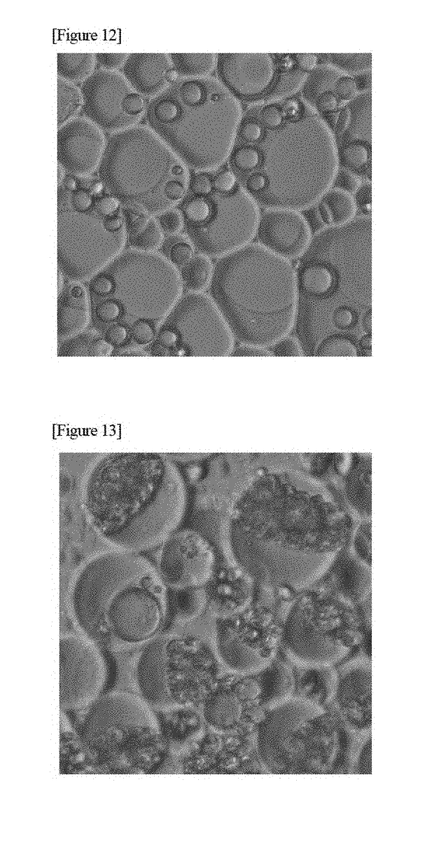

100 mg of a lactic acid-glycolic acid copolymer (Wako Pure Chemical Industries, Ltd.; LGA5020) and 100 mg of SUPPCIRE AM PASILLES (GATTEFOSSE) were dissolved in 1 mL of methylene chloride, and thereafter, 250 μL of an aqueous solution of 0.05% chitosan (Wako Pure Chemical Industries, Ltd.; Chitosan 100) and 0.5% acetic acid, containing 0.05% vitamin B12 (ALEXIS BIOCHEMICALS), was emulsified in the above-prepared solution at 20,000 rpm for 30 seconds, using a homogenizer (IKA ULTRA-TURRAX T18), to prepare a W/O emulsion. The prepared W/O emulsion was emulsified in 5 mL of 0.5% polyvinyl alcohol (Kuraray Co., Ltd.; KURARAY POVAL 220C) at 5000 rpm for 3 minutes, using a homogenizer (IKA ULTRA-TURRAX T18), to prepare a W/O/W emulsion. After completion of the preparation, the W/O/W emulsion was stirred using a stirrer, so that the solvent was distilled away. Thirty minutes later, the remaining emulsion was sampled, and was observed under an optical microscope. In some fine particles, a borderline was observed between the hemispheres thereof, and also, a state in which droplets were distributed in one hemisphere could be observed (FIG. 12). According to the observation under the optical microscope, the particle diameter of the Janus nanoparticles was found to be 1 to 100 μm.

### Example 12

100 mg of a lactic acid-glycolic acid copolymer (Wako Pure Chemical Industries, Ltd.; LGA5020), 100 mg of SUPPCIRE AM PASILLES (GATTEFOSSE) and 20 mg of monoglycerin stearate (Wako Pure Chemical Industries, Ltd.) were dissolved in 1 mL of methylene chloride, and thereafter, 250 μL of an aqueous solution of 0.05% chitosan (Wako Pure Chemical Industries, Ltd.; Chitosan 100) and 0.5% acetic acid, containing 0.05% vitamin B12 (ALEXIS BIOCHEMICALS), was emulsified in the above-prepared solution at 20,000 rpm for 30 seconds, using a homogenizer (IKA ULTRA-TURRAX T18), to prepare a W/O emulsion. The prepared W/O emulsion was emulsified in 5 mL of 0.5% polyvinyl alcohol (Kuraray Co., Ltd.; KURARAY POVAL. 220C) at 5000 rpm for 3 minutes, using a homogenizer (IKA ULTRA-TURRAX 118), to prepare a W/O/W emulsion. After completion of the preparation, the W/O/W emulsion was stirred using a stirrer, so that the solvent was distilled away. Thirty minutes later, the remaining emulsion was sampled, and was observed under an optical microscope. In some fine particles, a borderline was observed between the hemispheres thereof, and also, a state in which droplets were distributed in one hemisphere could be observed (FIG. 13). According to the observation under the optical microscope, the particle diameter of the Janus nanoparticles was found to be 1 to 100 μm.

### Example 13

1.0 g of chitosan (Wako Pure Chemical Industries, Ltd.; Chitosan 100) was dissolved in 500 mL of a 0.5% acetic acid aqueous solution, and 4500 mL of purified water was then added to the solution to prepare a chitosan solution. The prepared chitosan solution was subjected to spray drying using a spray drying device (PulvisGB22, YAMATO SCIENTIFIC CO., LTD.) under conditions consisting of an inlet temperature of 120° C., a drying air of 0.55 m²/min a pump scale of 0.5, and an atomizing air of 0.15 Mpa, so as to prepare chitosan fine particles (particle diameter according to observation under an electron microscope: 0.5 to 1 μm).

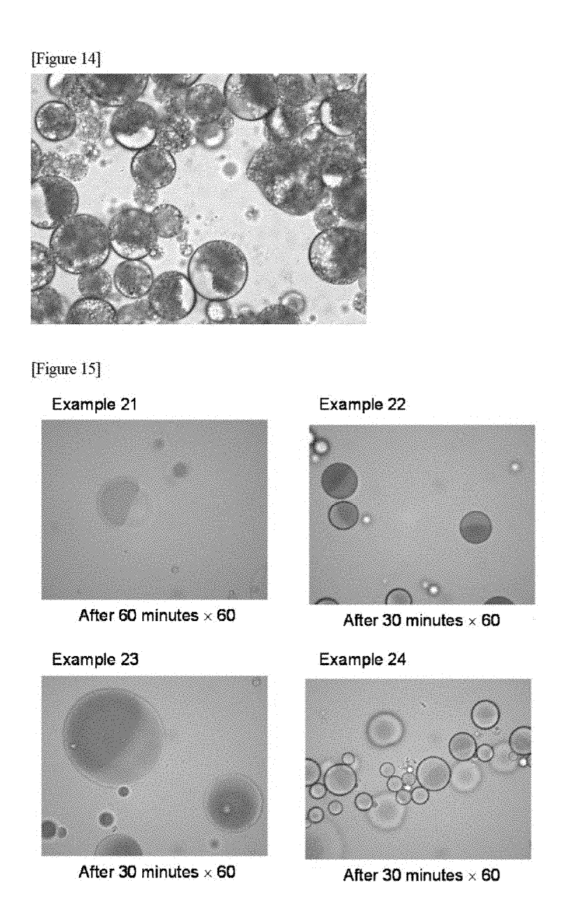

100 mg of a lactic acid-glycolic acid copolymer (Wako Pure Chemical Industries, Ltd.; LGA5020) and 100 mg of SUPPCIRE AM PASILLES (GATTEFOSSE) were dissolved in 1 mL of methylene chloride. Thereafter, 5 droplets of 5% oil red O-containing methylene chloride solution were added to the above-prepared solution, and 20 mg of the chitosan fine particles were then dispersed therein using a bath sonicator to prepare an S/O emulsion. The prepared S/O emulsion was emulsified in 5 of 0.5% polyvinyl alcohol (Kuraray Co., Ltd.; KURARAY POVAL 220C) at 5000 rpm for 3 minutes, using a homogenizer (IKA ULTRA-TURRAX T18), to prepare an S/O/W emulsion. The obtained emulsion was observed under an optical microscope, and as a result, the following was found (FIG. 14). 1: In some fine particles, a borderline was observed between the hemispheres thereof, 2: only one hemisphere was stained with oil red O; and 3: the chitosan fine particles were distributed in non-stained hemispheres. According to the observation under the optical microscope, the particle diameter of the Janus nanoparticles was found to be 1 to 100 μm.

### Examples 21 to 50 (Examples in Which Various Types of Lipids Were Used)

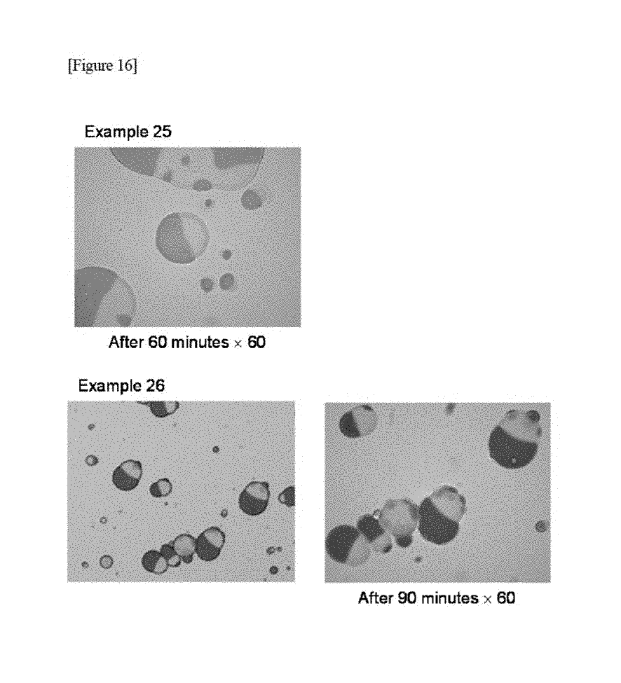

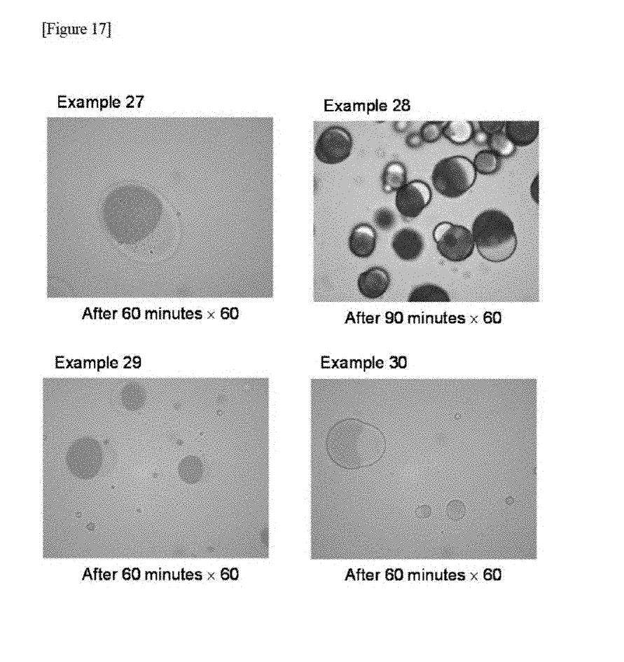

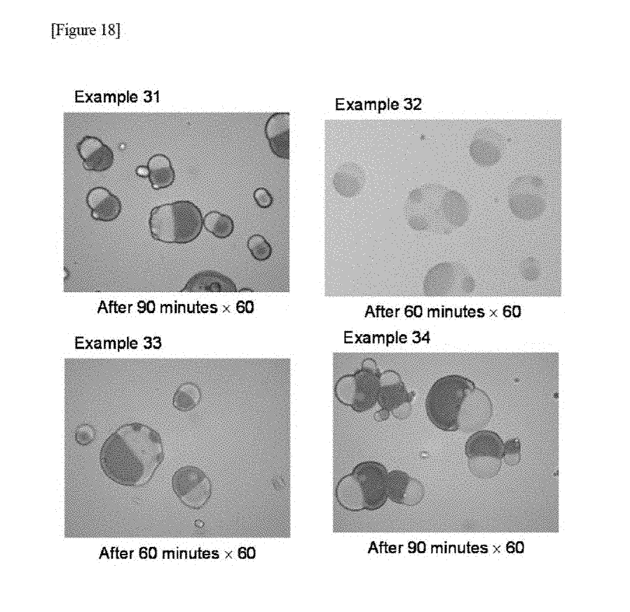

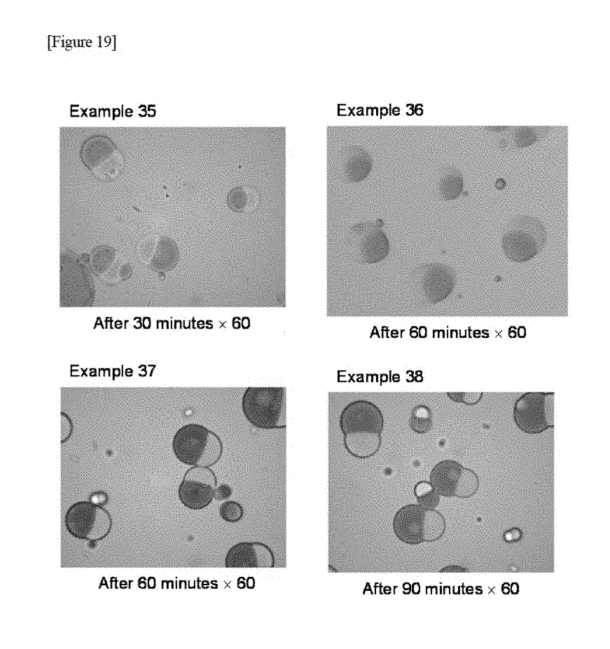

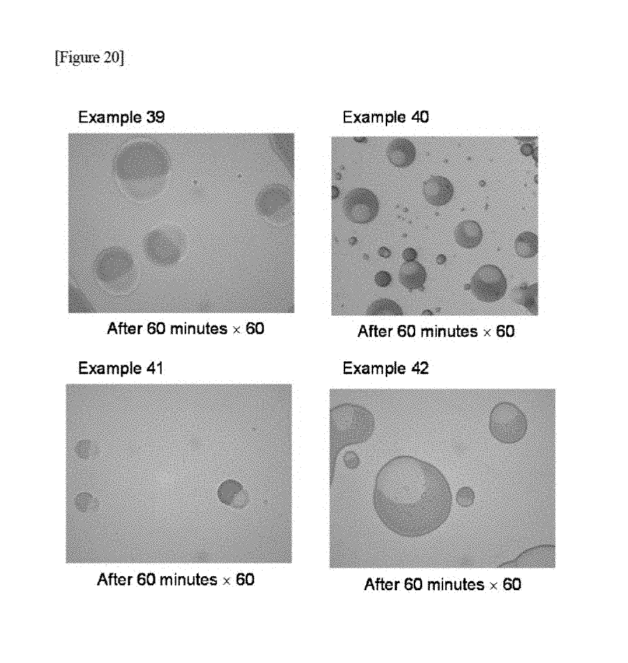

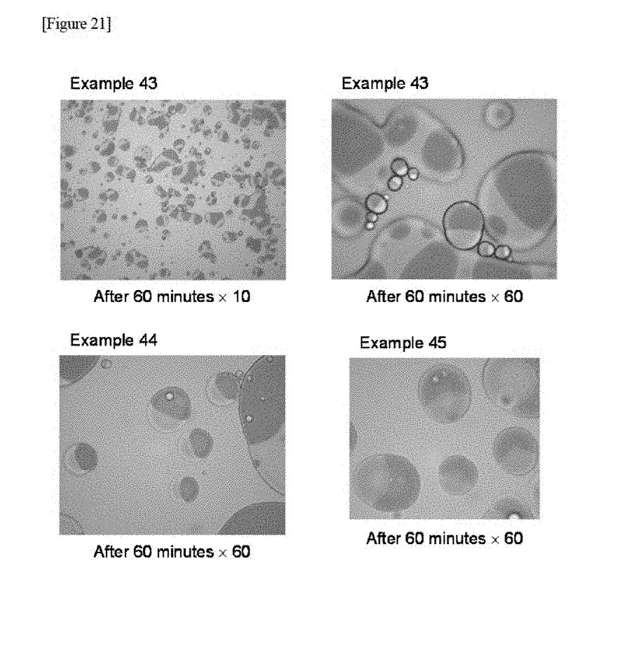

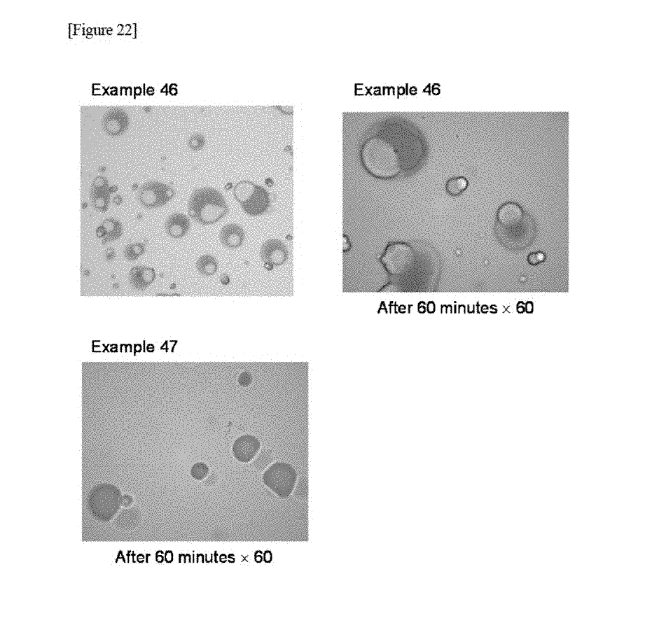

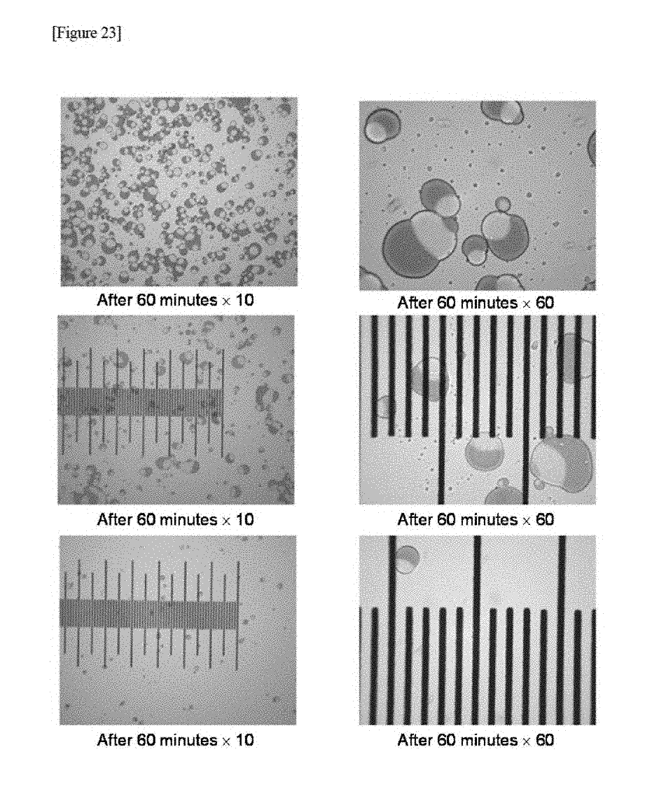

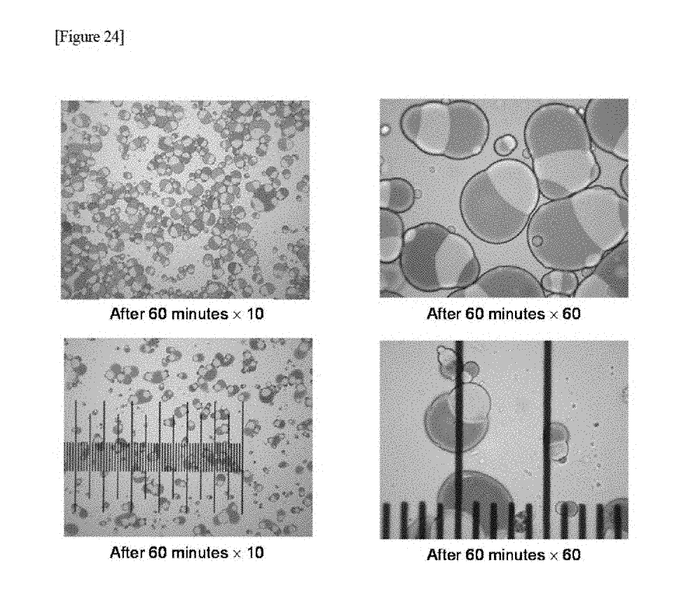

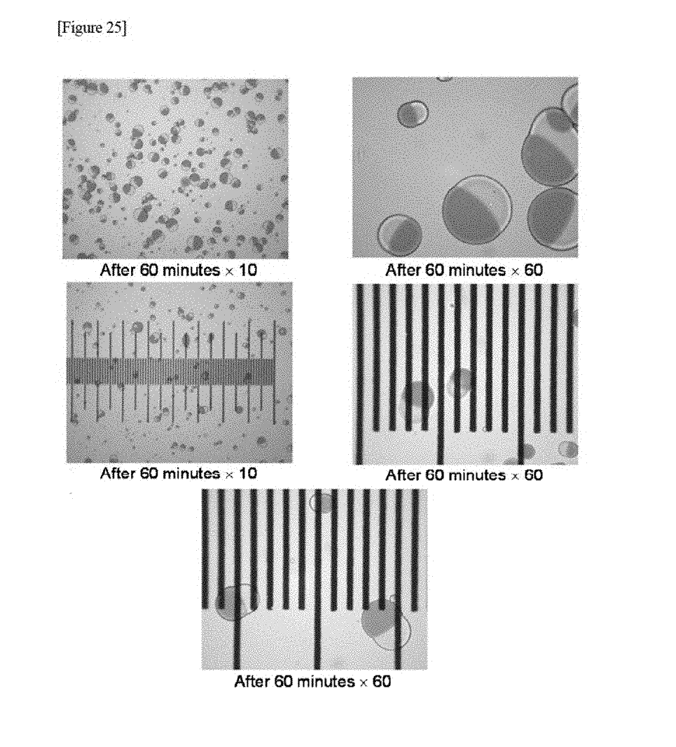

150 mg, of a lactic acid-glycolic acid copolymer (Wako Pure Chemical Industries, Ltd.; LGA5020) and 150 mg of each lipid shown in the following Table 1 were dissolved in 2 mL of methylene chloride, and the thus prepared solution was then emulsified in 5 mL of 0.5% polyvinyl alcohol (Kuraray Co., Ltd.; KURARAY POVAL 220C) at 5000 rpm for 3 minutes, using a homogenizer (ULTRA-TURRAX (registered trademark) T25 digital IKA (registered trademark), Model: T25DS1). After completion of the emulsification, the emulsion was stirred using a stirrer, so that the solvent was distilled away. After a predetermined period of time shown in the following Table 1, the remaining emulsion was sampled, and was observed under an optical microscope. In some fine particles, a borderline was observed between the hemispheres thereof (FIG. 15 to FIG. 25). According to the observation under the optical microscope, the particle diameter of the Janus nanoparticles in each of Examples 21 to 50 was found to be 1 to 100 μm.

### Example 51

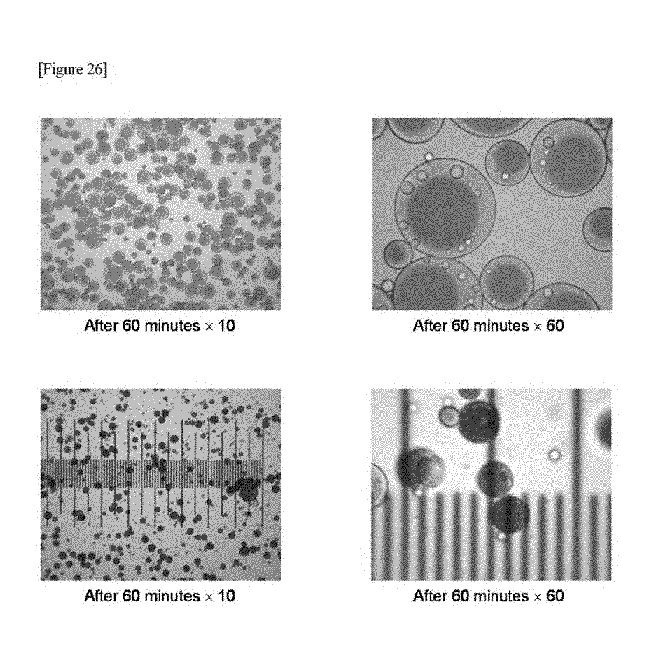

150 mg of polylactic acid (Wako Pure Chemical Industries, Ltd.; PL A0020) and 150 mg of SUPPOCIRE AM PELLETS (hydroxyl value: 5 mgKOH/g, melting point: 35.0° C. to 36.5° C.) (GATTEFOSSE) were dissolved in 2 mL of methylene chloride, and the thus prepared solution was then emulsified in 5 mL of 0.5% polyvinyl alcohol (Kuraray Co., Ltd.; KURARAY POVAL 220C) at 5000 rpm for 3 minutes, using a homogenizer (ULTRA-TURRAX (registered trademark) T25 digital IKA (registered trademark), Model: T25DS1). After completion of the emulsification, the emulsion was stirred using a stirrer, so that the solvent was distilled away. One hour later, the remaining emulsion was sampled, and was observed under an optical microscope. In some fine particles, a borderline was observed between the hemispheres thereof (FIG. 26). According to the observation under the optical microscope; the particle diameter of the Janus nanoparticles was found to be 1 to 100 μm.

### Example 52

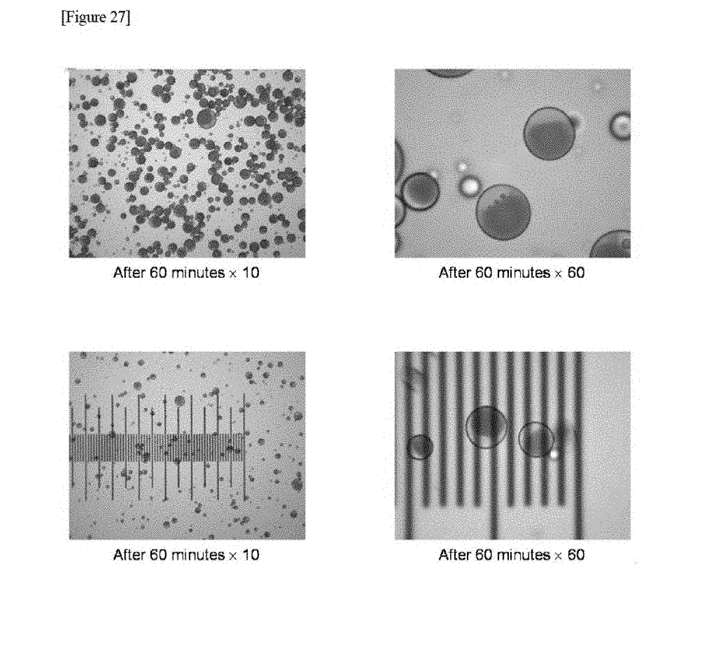

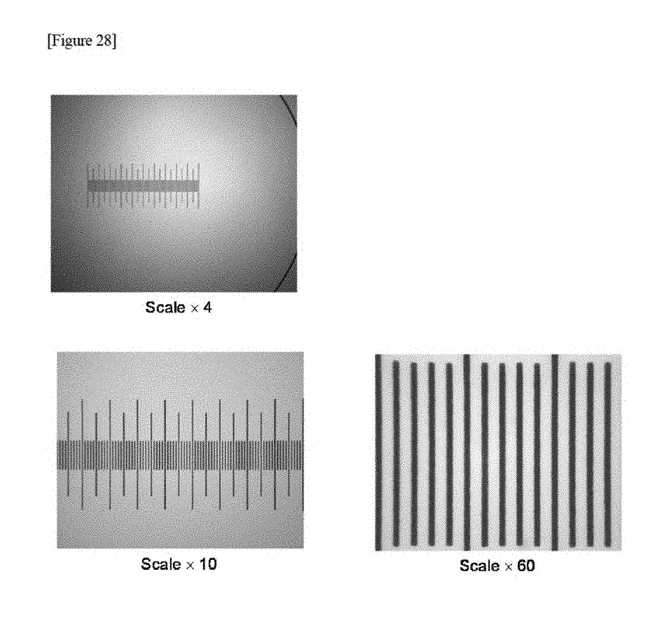

75 mg of polylactic acid (Wako Pure Chemical Industries, Ltd.; PLA0020), 75 mg of a lactic acid-glycolic acid copolymer (Wako Pure Chemical Industries, Ltd.; LGA5020), and 150 mg of SUPPOCIRE AM PELLETS (hydroxyl value: 5 mgKOH/g, melting point: 35.0° C. to 36.5° C.) (GATTEFOSSE) were dissolved in 2 mL of methylene chloride, and the thus prepared solution was then emulsified in 5 mL of 0.5% polyvinyl alcohol (Kuraray Co., Ltd.; KURARAY POVAL 220C) at 5000 rpm for 3 minutes, using a homogenizer (ULTRA-TURRAX (registered trademark) T25 digital IKA (registered trademark), Model:T25DS1). After completion of the emulsification, the emulsion was stirred using a stirrer, so that the solvent was distilled away. One hour later, the remaining emulsion was sampled, and was observed under an optical microscope. In some fine particles, a borderline was observed between the hemispheres thereof (FIG. 27). According to the observation under the optical microscope, the particle diameter of the Janus nanoparticles was found to be 1 to 100 μm.

TABLE 1

| | | Time after | | Melting | |
|---|---|---|---|---|---|
| | | solvent | | point of | Hydroxyl value of |
| | Lipid | distillation | | lipid (° C.) | lipid (mgKOH/g) |
| Example 21 | JAPOCIRE NAS 50 PELLETS | 1 | hour | 35.2 | 44 |
| | (GATTEFOSSE) | | | | |
| Example 22 | SUPPOCIRE BS2X PELLETS | 30 | minutes | 36.5 | 20 |
| | (GATTEFOSSE) | | | | |
| Example 23 | SUPPOCIRE NA PELLETS | 30 | minutes | 35.0 | 23 |
| | (GATTEFOSSE) | | | | |
| Example 24 | SUPPOCIRE NA 0 PELLETS | 30 | minutes | 35.1 | 2 |
| | (GATTEFOSSE) | | | | |
| Example 25 | SUPPOCIRE NAIS 10 PELLETS | 1 | hour | 38.9 | 12 |
| | (GATTEFOSSE) | | | | |
| Example 26 | JAPOCIRE NA 15 PELLETS | 1 | hour | 35.4 | 12 |
| | (GATTEFOSSE) | | | | |
| Example 27 | JAPOCIRE DM PELLETS (GATTEFOSSE) | 1 | hour | 44.0 | 4 |
| Example 28 | SUPPOCIRE DM PELLETS | 1.5 | hours | 44.0 | 4 |
| | (GATTEFOSSE) | | | | |
| Example 29 | JAPOCIRE NAS 50 PELLETS | 1 | hour | 35.2 | 44 |
| | (GATTEFOSSE) | | | | |
| Example 30 | SUPPOCIRE NA 15 PELLETS | 1 | hour | 35.1 | 12 |
| | (GATTEFOSSE) | | | | |
| Example 31 | SUPPOCIRE NA 10 PELLETS | 1.5 | hours | 35.9 | 10 |
| | (GATTEFOSSE) | | | | |
| Example 32 | SUPPOCIRE NSI 50 PELLETS | 1 | hour | 33.9 | 43 |
| | (GATTEFOSSE) | | | | |
| Example 33 | SUPPOCIRE NAI 25 PELLETS | 1 | hour | 35.3 | 24 |
| | (GATTEFOSSE) | | | | |
| Example 34 | SUPPOCIRE NAI 25A PELLETS | 1 | hour | 34.5 | 24 |
| | (GATTEFOSSE) | | | | |
| Example 35 | SUPPOCIRE NAS 40 PELLETS | 30 | minutes | 35.0 | 47 |
| | (GATTEFOSSE) | | | | |
| Example 36 | SUPPOCIRE NB PELLETS | 1 | hour | 37.0 | 23 |
| | (GATTEFOSSE) | | | | |
| Example 37 | SUPPOCIRE CM PELLETS | 1 | hour | 38.7 | 4 |
| | (GATTEFOSSE) | | | | |
| Example 38 | SUPPOCIRE AS2 PELLETS | 1.5 | hours | 35.6 | 20 |
| | (GATTEFOSSE) | | | | |
| Example 39 | SUPPOCIRE AS2X PELLETS | 1 | hour | 35.8 | 21 |
| | (GATTEFOSSE) | | | | |
| Example 40 | SUPPOCIRE BML PELLETS | 1 | hour | 36.3 | 4 |
| | (GATTEFOSSE) | | | | |
| Example 41 | SUPPOCIRE AP PELLETS | 1 | hour | 33.7 | 39 |
| | (GATTEFOSSE) | | | | |
| Example 42 | SUPPOCIRE AIML PELLETS | 1 | hour | 34.0 | 4 |
| | (GATTEFOSSE) | | | | |
| Example 43 | SUPPOCIRE BP PELLETS | 1 | hour | 35.9 | 40 |
| | (GATTEFOSSE) | | | | |
| Example 44 | SUPPOCIRE D PELLETS (GATTEFOSSE) | 1 | hour | 44.0 | 25 |
| Example 45 | SUPPOCIRE NBL PELLETS | 1 | hour | 36.6 | 25 |
| | (GATTEFOSSE) | | | | |
| Example 46 | SUPPOCIRE NAL PELLETS | 1 | hour | 34.8 | 24 |
| | (GATTEFOSSE) | | | | |
| Example 47 | SUPPOCIRE CS2X PELLETS | 1 | hour | 38.4 | 21 |
| | (GATTEFOSSE) | | | | |
| Example 48 | OVUCIRE 3460 PELLETS(GATTEFOSSE) | 1 | hour | 33.1 | 68 |
| Example 49 | OVUCIRE WL 3264 | 1 | hour | 33.9 | 43 |
| | PELLETS(GATTEFOSSE) | | | | |
| Example 50 | SUPPOCIRE BM PELLETS | 1 | hour | 36.4 | 3 |
| | (GATTEFOSSE) | | | | |

1. A method for producing a Janus nanoparticle having a particle diameter of 0.01 to 5000 μm, which is composed of a lipid and a polymer, wherein the method comprises:
a step of emulsifying a solution of one or more types of lipids and one or more types of polymers dissolved in a common solvent, in a liquid containing a surfactant; and
a step of removing the common solvent from the obtained emulsion.

2. A method for producing a Janus nanoparticle having a particle diameter of 0.01 to 5000 μm, which is composed of a lipid and a polymer, wherein the method comprises:
a step of dissolving or suspending one or two or more types of first surfactants in the oil phase and/or water phase of a solution of one or more types of lipids and one or more types of polymers dissolved in a common solvent, and then emulsifying the solution or suspension to prepare a W/O emulsion;
a step of emulsifying the obtained W/O emulsion in an aqueous solution of one or two or more types of second surfactants to prepare a W/O/W emulsion; and
a step of removing the common solvent from the obtained W/O/W emulsion.

3. The method according to claim 1, which is for use in controlling the distribution of an inner water phase in the Janus nanoparticle.

4. The method according to claim 1, wherein the solution of one or more types of lipids and one or more types of polymers dissolved in a common solvent comprises a drug.

5. The method according to claim 2, which uses an aqueous solution of a surfactant as a first surfactant(s), wherein the aqueous solution of the first surfactant(s) comprises a drug.

6. A method for producing a Janus nanoparticle having a particle diameter of 0.01 to 5000 μm, which is composed of a lipid and a polymer, wherein the method comprises:
a step of dispersing fine drug particles or particles formed by compounding one or more types of additives with a drug in a solution of one or more types of lipids and one or more types of polymers dissolved in a common solvent, to prepare an S/O emulsion;
emulsifying the obtained S/O emulsion in an aqueous solution of a surfactant(s) to prepare an S/O/W emulsion; and
a step of removing the common solvent from the S/O/W emulsion.

7. The method according to claim 4, wherein the drug is a peptide, a protein, a nucleic acid, or a nucleic acid derivative.

8. The method according to claim 1, wherein an aqueous solution of a surfactant(s) is used, and the volume of the aqueous solution of a surfactant(s) is 0.5 to 10 times greater than that of the solution of one or more types of lipids and one or more types of polymers dissolved in a common solvent.

9. The method according to claim 1, wherein the melting point of the lipid is 30° C. to 45° C.

10. The method according to claim 1, wherein the lipid is a glyceride base comprising saturated C8-C18 triglyceride fatty acid.

11. The method according to claim 1, wherein the polymer is a lactic acid-glycolic acid copolymer, polylactic acid, polyglycolic acid, or Eudragit (registered trademark) RS100.

12. The method according to claim 1, wherein the surfactant is a nonionic surfactant.

13. The method according to claim 1, wherein the surfactant is polyvinyl alcohol.

14. The method according to claim 1, wherein the common solvent is removed from the obtained emulsion over 30 minutes.

15. A Janus nanoparticle having a particle diameter of 0.01 to 5000 μm, composed of a lipid and a polymer, which is produced by the method according to claim 1.
